(19) United States
(12) Patent Application Publication
(10) Pub. No.: US 2019/0239913 A1
(43) Pub. Date: Aug. 8, 2019

(54) SURGICAL SHAVING INSTRUMENTS

(71) Applicant: GYRUS MEDICAL LIMITED, Cardiff (GB)

(72) Inventors: Ole SCHLOTTMANN, Cardiff (GB); David MORRIS, Rhondda (GB)

(73) Assignee: GYRUS MEDICAL LIMITED, Cardiff (GB)

(21) Appl. No.: 16/255,224

(22) Filed: Jan. 23, 2019

(30) Foreign Application Priority Data
Feb. 2, 2018 (GB) 1801739.2

(51) Int. Cl. A61B 17/32; A61B 18/14

(52) U.S. Cl. CPC A61B 17/32002; A61B 2018/00589; A61B 18/1482

(57) ABSTRACT

A surgical shaving instrument (1) comprises a main body (2), a hollow elongate shaft (4) extending from the main body (2). The elongate shaft (4) defines an elongate inner open volume through the shaft. A tip portion (18) extends from the distal end region of the elongate shaft (4). A driveshaft extends through the elongate shaft (4) from the main body (2) to the distal end region of the elongate shaft (4). A cutter head (12) is attached to the driveshaft and located in the tip portion (10), the cutter head (12) extends at least partially out of the tip portion (18). The tip portion (18) is of an electrically insulating ceramic material, and has an outer surface. An electrically conductive electrode is located on at least part of that outer surface.

The present invention relates to surgical shaving instruments, and, in particular, to arthroscopic surgical instruments.

### BACKGROUND OF THE INVENTION

Surgical shaving instruments are used by surgeons to perform surgical resection operations. Such instruments are also known bone burrs, or bone shavers. One particular type of instrument uses a rotating or oscillating cutting head mounted at a distal end of a hollow elongate shaft. The cutting head is typically driven by a motor or other drive means located in a device body from which the shaft extends. The cutting head is mounted on a driveshaft that extends through the elongate shaft. A distal tip portion of the elongate shaft provides a cutting window through which a portion of the cutting head extends for engagement with the tissue to be cut. Loose tissue (debris from the shaving action) can be removed from the cutting zone through the hollow elongate shaft, or by suction or some other means.

The distal tip portion can also be provided with, or define, a fixed cutting blade for manual cutting of tissue. The tip portion may also be provided with electrically conductive electrodes for supplying coagulating and ablation radio frequency (RF) energy to the targeted tissue. Typically, the elongate shaft and distal tip portion are of a metallic material. In the case of electrodes being provided at the distal tip, a thin layer of insulation is provided in order to insulate the electrical conductive electrode from the remainder of the distal end itself. The thin layer of insulation can limit the RF signal power that is able to be used. Accordingly, it is desirable to provide a design of instrument that allows the use of greater RF signal power.

### SUMMARY OF THE INVENTION

Aspects of the present invention are set out in the attached claims.

According to one aspect of the present invention, there is provided a surgical shaving instrument comprising a main body; a hollow elongate shaft extending from the main body, the shaft defining an elongate inner open volume which extends from a proximal end region to a distal end region of the shaft; a tip portion extending from the distal end region of the elongate shaft, the tip portion having an electrically insulating ceramic body defining a tip cavity therein, the electrically insulating ceramic body having an outer surface which defines an aperture therethrough into the tip cavity, such that the tip cavity is open; a driveshaft which extends through the inner open volume of the elongate shaft from the main body to the distal end region of the elongate shaft; and a cutter head attached to the driveshaft and located in the tip cavity of the tip portion, the cutter head extending at least partially out of the tip portion through the aperture, wherein an outer surface of the electrically insulating body and includes an electrically conductive electrode on at least part of that outer surface.

Such an instrument is able to provide a coagulation and/or ablation function in combination with a mechanical cutting function.

In one example, the electrode comprises a plurality of electrically independent electrodes.

In one example, the aperture of the tip portion is defined by an inner edge, and a portion of the inner edge defines a fixed shaver blade for the instrument. In one example, the shaver blade has a cutting angle in a range of 5 to 40°.

Such a fixed shaver blade provides for improved performance, and can result in a reduction of the size of the drive means needed to drive the cutter head.

One example further comprises drive means located in the main body for driving the cutter head via the driveshaft.

One example further comprises control means located in the main body for controlling drive of the cutter head.

One example further comprises control means located in the main body for providing radio frequency electrical signals to the or each electrode.

### BRIEF DESCRIPTION OF THE DRAWINGS

FIG. 1 is a schematic side view of a surgical shaving instrument;

FIG. 2 is a schematic cross-sectional side view of a distal end portion of the instrument of FIG. 1;

FIG. 3 is a schematic perspective view of a distal end portion of the instrument of FIGS. 1 and 2 embodying one aspect of the present invention;

FIG. 4 is a schematic perspective view of an alternative distal end portion design of the instrument of FIGS. 1 and 2 embodying one aspect of the present invention;

FIG. 5 is a schematic side view of the distal end portion of FIG. 3;

FIG. 6 is a simplified schematic side view of the distal end portion of FIG. 3 or 4; and

FIGS. 7A to 7C are schematic longitudinal cross-sectional end views of the distal end portion of FIG. 6 embodying another aspect of the present invention.

### DETAILED DESCRIPTION OF THE PREFERRED EMBODIMENTS

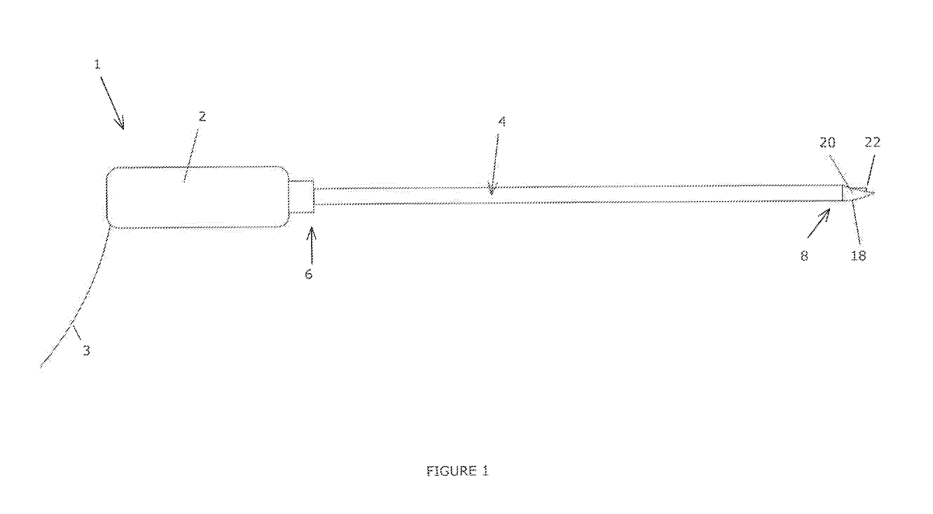

A surgical shaving instrument 1 is shown schematically in FIG. 1, and comprises a body 2 in which is located a drive unit and control means, for example control circuitry. The control means receives electrical power and control signals via a power and control connection 3, and may receive control signals from appropriately located control inputs, such as buttons or switches. Such control inputs may be located on the body 2, and/or in a suitable location for the user. The drive unit may include and electrical motor and/or oscillator.

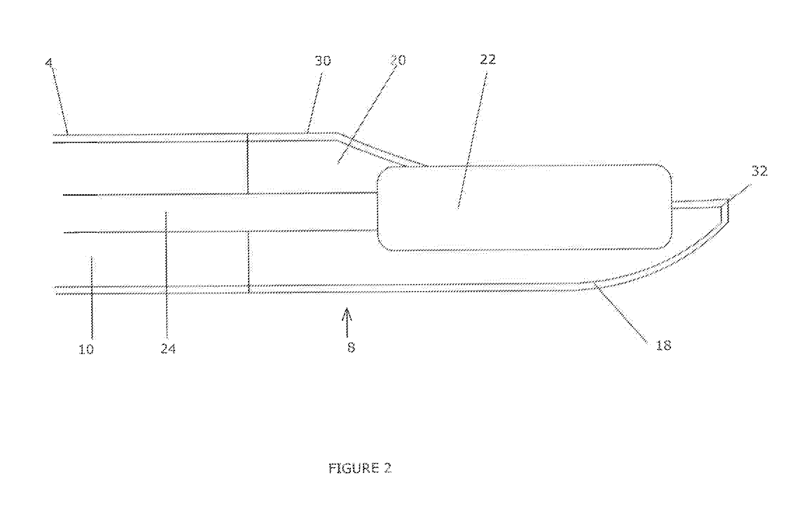

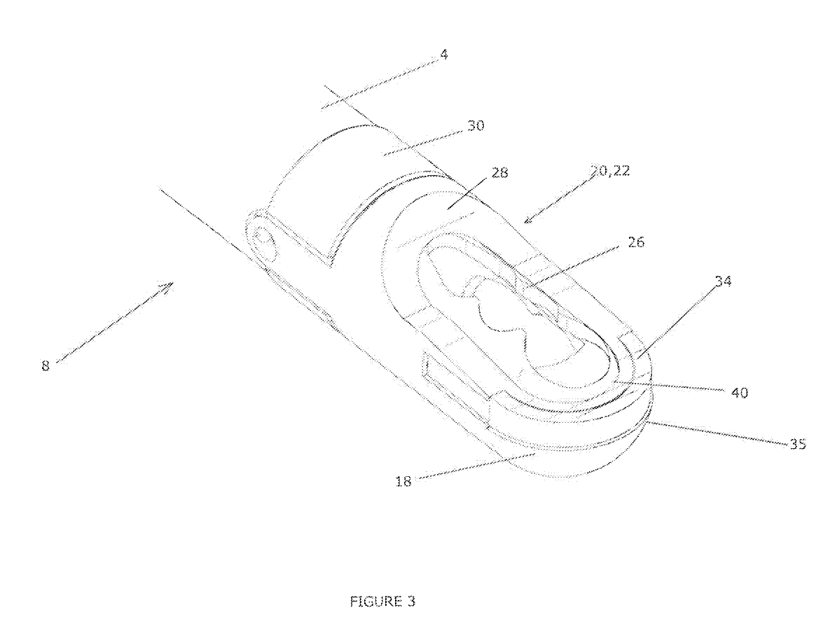

A distal end of the instrument of FIG. 1 is shown in more detail, in cross-sectional view, in FIG. 2, and in schematic perspective view in FIG. 3.

With reference to FIGS. 1 to 3, a hollow elongate shaft 4 extends from the body 2 from a proximal end region 6 to a distal end region 8 of the elongate shaft 4. The elongate shaft 4 extends along a longitudinal axis, and defines an inner open volume 10 therealong. The distal end region 8 carries a tip portion 18, which defines an inner tip cavity 20 contiguous with the inner open volume 10 of the elongate shaft 4. A cutting head 22 is located in the tip cavity 20. The cutting head 22 is driven by the drive unit of the body 2 via a driveshaft 24 that extends through the inner open volume 10 of the elongate shaft 4. The tip portion 18 defines an aperture 26 through a surface 28 of the tip portion 18. The surface 28 of the tip portion 18 that defines the aperture 26 is, in this example, a convex surface that extends from a first end region 30 of the tip portion 18 to a narrowed second end region 32 of the tip portion 32. The second end region 32 forms a distal tip of the instrument 1. The cutting head 12 extends at least partially through the aperture 26 so as to be able to contact tissue.

In one example, the driveshaft 24 is hollow, and defines an elongate lumen therealong. The cutting head 22 is also hollow, and is arranged to be contiguous with the lumen of the driveshaft 24. In use, tissue material removed by the cutting head 22 can be retrieved through the cutting head 22, and along the lumen of the hollow driveshaft 24, for removal.

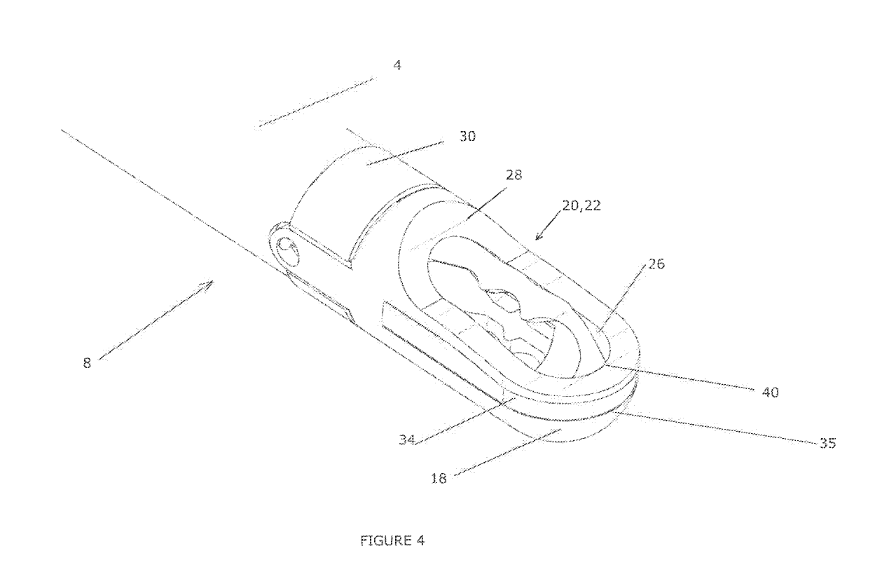

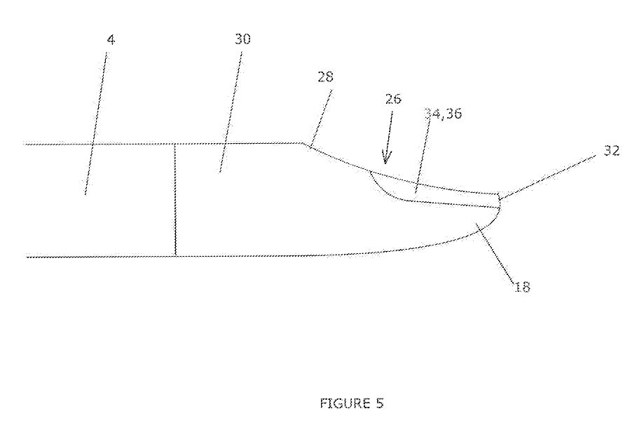

FIGS. 3 and 4 illustrate respective example designs of the end region 8 of an instrument embodying the present invention. FIG. 5 shows a simplified side view of the end portion 8. The tip portion 18 carries an electrically conductive electrode 34 on an outer region 35 thereof. This position for the electrode 34 allows for good contact with tissue being treated. In use, the electrode 34 is supplied with radio frequency electrical signals in order to provide coagulation and/or ablation function for the instrument 1. The radio frequency signals are provided from an RF generator, and supplied to the electrode 34 via the control circuitry in the body 2 of the instrument 1, in accordance with known practice for such coagulation and/or ablation functions. Detailed description of such function is omitted here for the sake of brevity.

In accordance with one aspect of the present invention, the tip portion 18 is of a ceramic non-electrically conductive (electrically insulating) material, such as an alumina, a zirconia, a carbide or a nitride. A non-electrically conductive material serves to insulate the electrode 34 from the metallic, electrically conductive elongate shaft 4, thereby enabling the provision of the radio frequency coagulation and ablation function provided by the electrodes 34 and 36. By manufacturing the tip portion 18 from an electrically insulating ceramic material, higher power radio frequency signals can be supplied to the electrode. Accordingly, coagulation and/or ablation performance can be improved compared with simply providing a thin insulating layer between the electrode 34 and the tip portion 18. Use of higher power radio frequency signals enables greater coagulation and/or ablation effectiveness.

The example shown provides a monopolar device with the electrode 35 being an active electrode and an electrical return path being provided by the patient. It will be readily appreciated that the electrode 35 may be provided by a pair of electrodes such that the device is a bipolar device, having a one active electrode and a return electrode. In such an example, such multiple electrodes are electrically insulated from one another. Another example embodiment includes more than two electrodes, such as three or more electrodes. Such a plurality of electrodes may be controlled in an appropriate manner. In one example having three electrodes, there may be two return electrodes and a single active electrode, or vice versa.

The tip portion 18 defines a fixed shaver blade 40 on a distal edge region of the aperture 26 through the surface 28. This fixed shave blade 40 is used by the surgeon for manual removal of tissue, sometimes to be broken up and removed by the cutter head 12. The fixed shaver blade 40 is directed towards the elongate shaft 4 from the distal end of the tip portion 18 so that cutting is ached by pulling of the instrument 1 towards the operator. The manufacture of the tip portion 18 of a ceramic material serves to ensure that the fixed shaver blade 40 is more durable (from the edge perspective) than if provided by a metallic material.

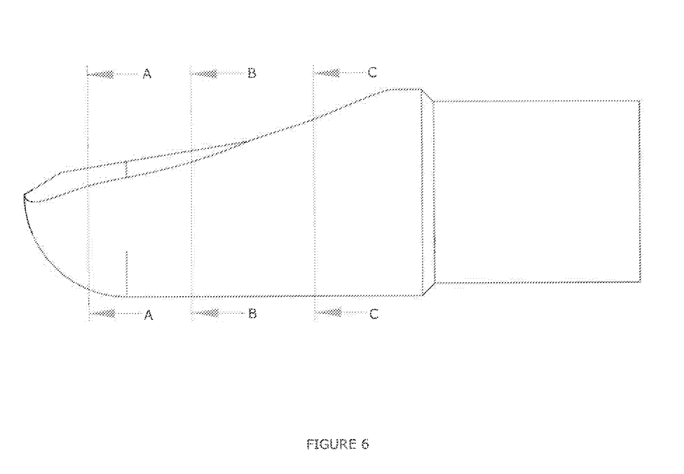

FIG. 6 illustrates a schematic simplified side view of the end region 8. As before, the tip portion 18 has a first end region 30 that extends from the shaft 4. The tip portion 18 defines a fixed shaver blade 40. In accordance with another aspect of the present invention, the fixed shaver blade is defined by an inner edge of the ceramic tip portion 18.

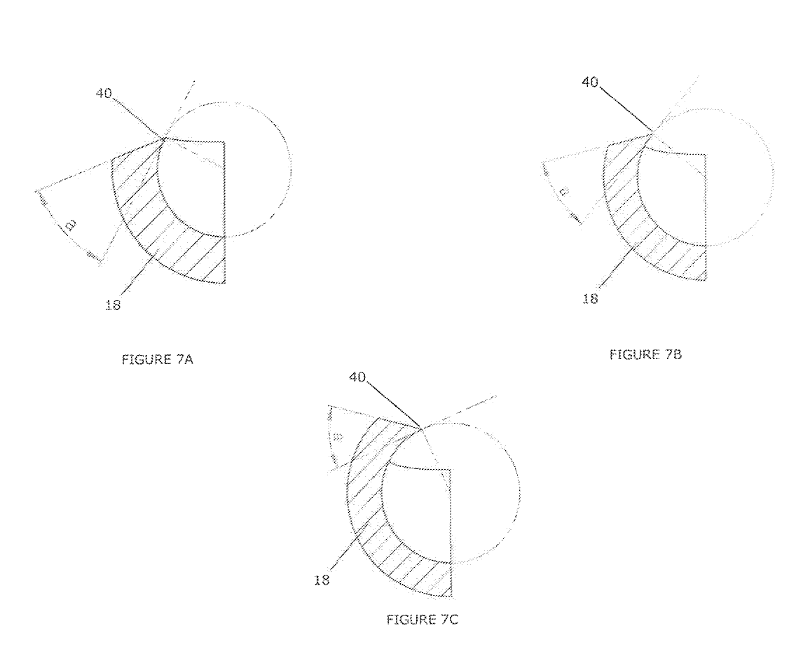

FIGS. 7A, 7B and 7C show respective longitudinal cross sections taken along section lines A-A, B-B, and C-C, and illustrate how the upper surface angle changes along the length of the tip portion 18, so that the cutting edge angle a of the fixed shaver blade remains substantially constant along that length.

The fixed shaver blade 40 has a cutting edge having an acute cutting angle a, for example in the range of 5° to 40° depending on the requirements of the instrument. This sharper fixed shaver blade 40 allows for the reduction of cutting speed, torque and power requirements of the cutter head 12, which results in the ability to provide a smaller drive unit in the body 2 of the instrument 1. This size reduction then allows the instrument to be reduced in size, and may then be provided as a one-piece instrument.

1. A surgical shaving instrument comprising:
a main body;
a hollow elongate shaft extending from the main body, the shaft defining an elongate inner open volume which extends from a proximal end region to a distal end region of the shaft;
a tip portion extending from the distal end region of the elongate shaft, the tip portion having an electrically insulating ceramic body defining a tip cavity therein, the electrically insulating ceramic body having an outer surface which defines an aperture therethrough into the tip cavity, such that the tip cavity is open;
a driveshaft which extends through the inner open volume of the elongate shaft from the main body to the distal end region of the elongate shaft; and
a cutter head attached to the driveshaft and located in the tip cavity of the tip portion, the cutter head extending at least partially out of the tip portion through the aperture, wherein an outer surface of the electrically insulating ceramic body includes an electrically conductive electrode on at least part of that outer surface.

2. A surgical shaving instrument as claimed in claim 1, wherein the electrode comprises a plurality of electrically independent electrodes.

3. A surgical shaving instrument as claimed in claim 1, wherein the driveshaft defines an elongate lumen therethrough.

4. A surgical shaving instrument as claimed in claim 1, wherein the aperture of the tip portion is defined by an inner edge, and a portion of the inner edge defines a fixed shaver blade for the instrument.

5. A surgical shaving instrument as claimed in claim 3, wherein the shaver blade has a cutting angle substantially in a range of 5° to 40°.

6. A surgical shaving instrument as claimed in claim 3, wherein the shaver blade has a cutting angle which is substantially constant along the inner edge.

7. A surgical shaving instrument as claimed in claim 1, further comprising drive means located in the main body for driving the cutter head via the driveshaft.

8. A surgical shaving instrument as claimed in claim 7, wherein the drive means is operable to oscillate the driveshaft.

9. A surgical shaving instrument as claimed in claim 7, wherein the drive means is operable to rotate the driveshaft.

10. A surgical shaving instrument as claimed in claim 1, further comprising control means located in the main body for controlling drive of the cutter head.

11. A surgical shaving instrument as claimed in claim 1, further comprising control means located in the main body for providing radio frequency electrical signals to the or each electrode.
